(19) United States
(12) Patent Application Publication
(10) Pub. No.: US 2008/0123106 A1
(43) Pub. Date: May 29, 2008

(54) Surface Roughness Measurement Methods and Apparatus

(75) Inventors: Haishan Zeng, Vancouver (CA); Lioudmila Tchvialeva, Vancouver (CA); Tim K. Lee, Burnaby (CA); David I. McLean, Vancouver (CA); Harvey Lui, Vancouver (CA)

Correspondence Address: OYEN, WIGGS, GREEN & MUTALA LLP;480 - THE STATION, 601 WEST CORDOVA STREET, VANCOUVER, BC, V6B 1G1, omitted

(73) Assignee: BC CANCER AGENCY, Vancouver, BC (CA)

(21) Appl. No.: 11/722,878

(22) PCT Filed: Dec. 23, 2005
(86) PCT No.: PCT/CA05/01967

§ 371 Date: Jun. 26, 2007

Related U.S. Application Data
(60) Provisional application No. 60/638,399, filed on Dec. 27, 2004.

(51) Int. Cl. G01B 11/30; G01B 11/02; G06F 19/00; A61B 5/00

(52) U.S. Cl. 356/600; 702/172; 702/97; 600/306

(57) ABSTRACT

Surface roughness measurements are made by illuminating a surface with coherent light to generate a speckle pattern and studying characteristics of the speckle pattern. The disclosed techniques may be applied to measuring the surface roughness of skin or other biological surfaces. Skin roughness information may be used in the diagnosis of conditions such as malignant melanoma. Methods and apparatus for measuring the coherence length of optical sources involve extracting information about speckle patterns resulting when light from the optical sources interacts with a surface having a known roughness.

### CROSS-REFERENCE TO RELATED APPLICATION

This application claims priority from U.S. 60/638,399 filed on 27 Dec. 2004 entitled APPARATUS AND METHODS RELATING TO THE DETECTION AND ANALYSIS OF OPTICAL SPECKLE, which is hereby incorporated herein by reference. For purposes of the United States, this application claims the benefit of U.S. 60/638,399 under 35 U.S.C. §119.

### TECHNICAL FIELD

The invention relates to measuring the roughness of surfaces. Embodiments of the invention may be applied to make measurements of the surface roughness of skin and other biological surfaces. Such measurements may be useful in the diagnosis of cancer or other skin conditions. The invention also relates to the measurement of coherence length in optical radiation.

### BACKGROUND

Surface finish can be important in manufacturing. There exist various technologies for measuring the roughness of surfaces. Mechanical profilometers are one type of surface roughness measuring instrument. A mechanical profilometer has a stylus that is dragged across a surface. The stylus follows contours of the surface. The surface roughness is evaluated by monitoring the motion of the stylus. Other techniques that have been applied for the measurement of surface roughness include:
Optical profilometry based on the detection of reflected light, which depends on the depth, and the angles of skin relief;
Laser profilometry based on dynamic focusing of a laser beam onto a specimen replica. The height of a point on the surface of the replica is deduced from the setting of a focusing lens;
Interference fringe profilometry based on calculating a phase image from an interference fringe pattern. The phase image gives access to the altitude of each point of a surface replica; and,
Electro-mechanical devices such as piezoelectric probes or arrays of micro-sensors may be used to detect surface profiles.

U.S. Pat. No. 5,748,311 discloses a method and system for measuring geometric properties of single rough particles. A volume of fluid containing the particles is illuminated with coherent radiation to yield a distribution of scattered radiation having a speckle structure. The distribution is detected with a one-dimensional or two-dimensional image detector. The surface roughness of a particle under investigation is estimated from the contrast of the measured intensity distribution.

U.S. Pat. No. 3,804,521 discloses an optical device for characterizing the surface roughness of a sample. A source of spatially coherent light having a wide spectral bandwidth is directed at the surface. Light scattered from the surface is imaged onto a single-channel light detector. The image is scanned by moving the sample or by moving a pinhole to determine the speckle contrast of the image. The surface roughness is estimated from the speckle contrast.

U.S. Pat. No. 4,145,140 discloses a method and apparatus for measuring surface roughness using statistical properties of dichromatic speckle patterns. The method involves illuminating a surface with spatially coherent light of at least two wavelengths and analyzing speckle patterns formed by light at each of the wavelengths.

U.S. Pat. No. 4,334,780 discloses an optical method for evaluating surface roughness of a specimen. The method involves illuminating a surface with a laser beam, imaging scattered light with a transform lens, and measuring light distribution half widths.

U.S. Pat. No. 5,293,215 discloses a device for interferometric detection of surface structures by measurement of the phase difference in laser speckle pairs.

U.S. Pat. No. 5,608,527 discloses an apparatus for measuring surface roughness of a surface that includes a multi-element array detector positioned to receive specular light reflected by the surface and light that has been scattered from the surface.

Optical surface measurement systems which monitor characteristics of specular light reflected from a surface being studied are disclosed in U.S. Pat. No. 5,162,660, U.S. Pat. No. 4,511,800, U.S. Pat. No. 4,803,374 and U.S. Pat. No. 4,973,164.

Surface roughness is a criteria that can be used in assessing the status of human skin. According to the classification given in K., Hashimoto. New Methods for Surface Ultrastructure. Comparative Studies of Scanning Electron Microscopy, Transmission Electron Microscopy and Replica Method. Int. J. Dermatol. 82 (1974) pp. 357-381, the surface pattern of human skin can be divided into:
a primary structure of macroscopic, wide, deep (20-100 μm) lines or furrows;
a secondary structure of finer, shorter and shallower (5-40 μm) secondary lines or furrows running over several cells; and,
a tertiary structure made up of lines having depths on the order of (0.5 μm) that are the borders of individual horny cells of the skin.
   The primary and secondary lines form a topological map of the skin. The map has a net-like structure and consists of polygonal forms, most often triangles.

Many profilometric techniques are not practically usable for measuring the roughness of skin in vivo due to a combination of inaccuracy, poor reproducibility, complexity, and cost. Various attempts to measure the surface roughness of human skin in vivo have produced disappointing results. It has been common to make replicas of a subject's skin surface and to measure the surface roughness of the replicas. However, making a replica is a highly operator-dependent procedure and may produce a variety of artifacts. An imperfect replica can have a microtopography that is significantly different from the skin that it attempts to replicate.

Papers that discuss the quantitative analysis of skin topography include:
Ma'or Z. et al. Skin smoothing effects of Dead Sea minerals: comparative profilometric evaluating of skin surface. Int J. Cosm. Sci 19, 105-110 (1997);
Bourgeois, J. F. et al. Radiation-induced skin fibrosis after treatment of breast cancer: profilometric analysis. Skin Research and Technology 9 (1), 39-42 (2003).

Lagarde, J. M. et al. Skin topography measurement by interference fringe projection: a technical validation. Skin Research and Technology 7 (2), 12-121 (2001) and Tanaka, et al. The “Haptic Finger”—a new device for monitoring skin condition. Skin Research and Technology 9 (2), 131-136 (2003) disclose attempts to measure skin roughness in vivo.

US 20040152989 discloses a system for measuring biospeckle of a specimen. The system includes a source of coherent light, such as a laser, capable of being aimed at a specimen; a camera capable of obtaining images of the specimen; and a processor coupled to the camera. The processor has software capable of performing bio-activity calculations on the plurality of images. The bio-activity calculations may include a Fourier Transform Analysis, Power Spectral Density, Fractal Dimensional Calculation, and/or Wavelet Transform Analysis.

WO1999044010 and U.S. Pat. No. 6,208,749 disclose a digital imaging method for measuring multiple parameters from an image of a lesion, one of which is texture.

Skin texture features, based on the second-order statistics, have been used as aides in differentiating malignant skin tumours (melanoma) from benign tumours (seborrheic keratosis) as described in Deshabhoina, Srinivas V. et al. Melanoma and seborrheic keratosis differentiation using texture features. Skin Research and Technology 9 (4), 348-356. (2003).

Malignant melanoma (MM) is the most aggressive skin cancer and is consistently lethal if left untreated. MM removal at early stages is usually curative. Therefore, early detection of MM is very important. There are some difficulties in MM diagnostics because benign pigmented skin lesions (PSL) like seborrheic keratosis (SK) and pigmented nevi (PN) resemble melanoma. Clinical diagnostic sensitivity (the proportion of all cases of histologically proven MM that were diagnosed as MM) differs: 80% for trained dermatologists and approximately 40% for nondermatologists. A main goal of new diagnostics techniques is to increase the sensitivity of diagnostics for MM and other similar conditions.

It is also desirable to minimize the excision of benign lesions. A large proportion of biopsies taken by nondermatologists of suspected malignant skin lesions have been found to be benign. To avoid unsuitable surgery the diagnostics specificity (the proportion of all cases not proven histologically to be MM that was diagnosed as ‘not-melanoma’) should be pressed toward higher values. Therefore, there is an ongoing need for rapid, noninvasive, accurate technique that can be utilized for characterization of skin lesions prior to invasive biopsy.

MM and similar conditions can be diagnosed based on subjective evaluation by trained clinicians. Clinicians analyze lesion images obtained by techniques including examination with the naked eye. The current practice in melanoma diagnosis is based on the ABCD rule, which uses four simple clinical morphological features that characterize melanoma lesions (Asymmetry, Border irregularity, Color variegation, and Diameter of more than 5 mm). However clinical diagnosis based on the ABCD rule has only 65% to 80% sensitivity and 74-82% specificity. This is largely because this method does not recognize that small melanomas (less than 5 mm) may occur. In addition, very early melanomas may have a regular shape and homogeneous color; such lesions would falsely be assessed as benign. Another problem is that the ABCD rule can misidentify some benign PN as melanoma.

Epiluminescent microscopy (also termed dermoscopy, skin surface microscopy, dermatoscopy) involves covering the skin lesion with mineral oil, alcohol, or even water and then inspecting the lesion with a hand-held scope (also called a dermatoscope), a stereomicroscope, a camera, or a digital imaging system. Some dermatoscopes have polarized light sources and do not require that a fluid be placed on a lesion that is being inspected. It has been reported that epiluminescent microscopy allows trained specialists to achieve a diagnostic accuracy rate better than inspection with the naked eye.

Other techniques such as sonography, thermography, Raman spectroscopy, near infrared spectroscopy and confocal scanning laser microscopy have also been found to be useful in diagnosis of MM. In the last decade, numerous automatic diagnostic systems have been developed. These systems have attempted to diagnose MM automatically based on various physical phenomena. Researchers are still seeking image parameters and classification rules that can be used to automatically diagnose MM. Despite many attempts, a noninvasive, rapid, reliable method for MM diagnosis has not yet been established.

U.S. Pat. No. 6,008,889 discloses apparatus for diagnosis of a skin disease site using spectral analysis. The apparatus includes a light source for generating light to illuminate the disease site and a probe unit optically connected to the light source for exposing the disease site to light to generate fluorescence and reflectance light.

Despite the work that has been done in this field there remains a need for practical and cost-effective systems and methods for measuring surface roughness. In the medical arts, there is a particular need for systems and methods capable of measuring the roughness of areas of skin in vivo.

### SUMMARY OF THE INVENTION

This invention has various aspects. One aspect of the invention provides methods for measuring the roughness of biological surfaces such as skin, the surfaces of internal organs, or the like. The methods involve making measurements of speckle patterns produced by the scattering of coherent optical radiation from the biological surfaces. In some embodiments, the methods are performed on biological surfaces in vivo. Such methods may comprise: illuminating an area of a biological surface of a subject with coherent optical radiation and allowing the optical radiation to scatter from the area of the biological surface to yield a speckle pattern; making measurements of intensity of the optical radiation in the speckle pattern; and, based upon results of the measurements, computing a measure of roughness of the area of the biological surface.

Another aspect of the invention provides apparatus for measuring the roughness of a biological surface. The apparatus comprises a light source emitting optical radiation having a coherence length of 300 μm or less; an imaging detector located to detect the optical radiation after the optical radiation has been scattered from a biological surface; and, a processor connected to receive image data from the imaging detector. The processor is configured to: compute a contrast of a speckle pattern in the scattered optical radiation; and, compute a roughness of the biological surface from the contrast.

A further aspect of the invention provides a method for evaluating a coherence length of optical radiation. The method is performed using a programmed computer and comprises: directing the optical radiation at a surface having a known roughness to yield a speckle pattern; determining a contrast of the speckle pattern; and, computing the coherence length of the optical radiation from the contrast of the speckle pattern.

Further aspects of the invention and features of specific embodiments of the invention are described below.

### BRIEF DESCRIPTION OF THE DRAWINGS

In drawings which illustrate non-limiting embodiments of the invention,

FIG. 1 is a schematic view of optical apparatus for measuring surface roughness of skin in which an area of skin is illuminated by light having a substantially continuous spectrum over a range of wavelengths;

FIG. 1A is a schematic view of apparatus according to an alternative embodiment of the invention;

FIG. 2 is an example speckle pattern of the type that could be obtained using the apparatus of FIG. 1;

FIG. 3 is a theoretical curve showing speckle pattern contrast as a function of roughness times spectral line width for sandpaper samples;

FIG. 4 shows linear and angular profiles of a speckle pattern as can arise from spatial incoherence;

FIG. 5 illustrates contrast as a function of radial distance of speckle patterns created by shorter- and longer-coherence-length light sources;

FIGS. 6A and 6B show one-dimensional autocorrelation for speckle patterns imaged at spot sizes of 3 mm and 2 mm respectively;

FIG. 7 illustrates reflection of light from layers on a surface to create independent speckle patterns;

FIG. 8 is a plot showing speckle pattern contrast measured using apparatus like that of FIG. 1 as a function of surface roughness for a number of surfaces;

FIG. 9 illustrates apparatus according to an alternative embodiment of the invention;

FIG. 10 illustrates apparatus according to another alternative embodiment of the invention; and,

FIG. 11 is a flow chart illustrating a method for measuring skin roughness according to the invention.

All of the appended drawings of apparatus are schematic in nature. In those drawings, certain features have been shown in greatly exaggerated or diminished scales for purposes of illustration.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

This invention relates to the measurement of roughness of surfaces. The invention will be described using, as a primary example, the measurement of skin roughness in vivo. Skin roughness measurements can be of assistance in:
diagnosis of various conditions including some cancers (for example, skin roughness is a factor that can be used to distinguish between malignant melanomas and other conditions such as seborrheic keratosis);
assessing the efficacy and progress of dermatological or cosmetic treatments;
assessing skin dryness and wrinkling;
assessing skin roughness resulting from xerosis, aging and photoaging; and
monitoring how skin roughness changes in response to therapy for such conditions.
   Various aspects of the invention may be applied to the measurement of surface roughness in other contexts. A number of new and inventive methods and apparatus for measuring surface roughness are described herein. Also described herein are methods and apparatus for measuring the line width of coherent light.

All of the techniques described herein measure surface roughness by creating speckle patterns and measuring characteristics of the speckle patterns. The application of such techniques to measuring the roughness of skin and other biological surfaces, such as the surfaces of internal organs, in vivo is considered to be novel and inventive. Speckle can be regarded as an interference pattern produced by coherent light scattered from different parts of an illuminated surface. The intensity of light observed at each point in a speckle pattern is the result of the sum of many elementary light waves. Each of the elementary light waves has a stochastic phase.

If the illuminated surface is rough on the scale of the wavelength of the illuminating light, elementary light waves reflected from different points on the surface will traverse different optical path lengths in reaching any point in space where speckle can be observed. The resulting intensity at the point will be determined by coherent addition of the complex amplitudes associated with each of these elementary waves. If the resultant amplitude is zero, or near zero, a “dark speckle” will be formed, whereas if the elementary waves are in phase at the point, an intensity maximum will be observed at the point and a “bright speckle” will be formed.

A useful speckle pattern cannot be observed in cases where the coherence length of the illuminating light is either much less than or much greater than the roughness of the surface. Speckle patterns can be observed in cases where the coherence length of the illuminating light is comparable with the roughness of the surface.

Using speckle patterns to characterize the roughness of a surface can be advantageous because speckles are formed as a result of illumination of an entire illuminated surface. A speckle pattern inherently averages information about points over the entire surface. Therefore measurements made on speckle patterns can be statistically significant, reliable, and repeatable.

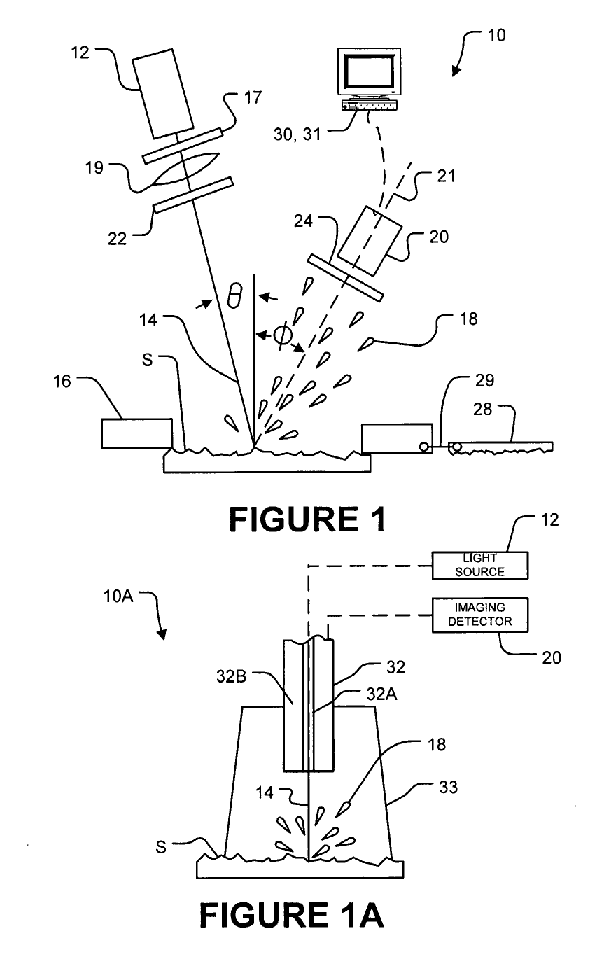

FIG. 1 is a schematic view of apparatus 10 according to an example embodiment of the invention. Apparatus 10 measures surface roughness by measuring the contrast of a speckle pattern. Apparatus 10 comprises a light source 12 that emits a beam 14 of light having a spectrum that includes a range of wavelengths between wavelengths λ₁ and λ₂. The spectrum is preferably substantially continuous in the range of λ₁ to λ₂ Light source 12 may comprise, for example, a laser, a fibre-coupled diode laser; a light-emitting diode (LED); a super luminescent diode (SLD or SLED); or another light source.

In some embodiments, light source 12 comprises a light-emitting diode LED combined with a narrow-band filter, typically an interference filter, to provide a beam having the desired spectral characteristics. In some embodiments the LED is a green-emitting or blue-emitting LED. For example, the LED could be:
A green-emitting LED, such as a ETG model ETG-5XB527-30 LED that emits primarily green light with a dominant wavelength of 529 nm; or
A blue-emitting LED such as a model LXHL-LR5C available from Lumileds Lighting, USA that emits primarily blue light having a wavelength of 455 nm and has a bandwidth of 20 nm.
   Such a LED may be combined with a narrow-band filter such as an interference filter, if necessary, to provide a bandwidth on the order of 10 nm. The bandwidth may be, for example in the range of 5 to 50 nm to provide coherence lengths suitable for measurements of surface roughness in certain ranges. The coherence length of light source 12 may be adjustable to permit measurements of different ranges of surface roughness. This may be achieved, for example, by providing a light source comprising an LED and a series of narrow-band filters having different bandwidths.

In a prototype embodiment, light source 12 comprises a 10.66 mW fiber-coupled diode laser emitting light at wavelength of approximately 658 nm filtered by a diaphragm 17 and collimated by a collecting lens 19 to form a beam 14.

Light source 12 emits light having a coherence length comparable to the surface roughness of a surface being investigated. For example, where the surfaces of interest have surface roughness in the range of 10 μm to 100 μm the coherence length of the light in beam 14 should be comparable to 10 μm to 100 μm (e.g. for measuring the roughness of surfaces having a roughness on the order of 10 μm the coherence length of the light in beam 14 should be less than about 250 μm and preferably in the range of about 25 μm to about 250 μm). From Equation (7) below it can be shown that providing in apparatus 10, a beam 14 having a coherence length of 200 μm permits measurement of surface roughnesses in the range of about 7.5 μm≦σ≦75 μm.

The coherence length is related to the difference between λ₁ and λ₂ by the relationship:

$${L}_{c} = \frac{{λ}^{2}}{\vert {λ}_{2} - {λ}_{1} \vert} \qquad ( 1 )$$

where λ is the wavelength midway between λ₁ and λ₂.

The width of beam 14 is selected to provide an area of illumination that will yield speckles of a convenient size. Beam 14 may, for example, have a diameter in the range of about 1 mm to 5 mm. In a prototype embodiment, beam 14 had a width set to either 2 mm or 3 mm.

Beam 14 is directed onto an area S of a subject's skin (or some other surface having a surface roughness to be measured). In the illustrated embodiment, light source 12 is fixed relative to a support plate 16 that beam 14 is incident on area S with a known geometry. In the illustrated embodiment, beam 14 is incident on area S at an angle θ to a normal to area S. Angle θ is preferably small, for example, about 5 degrees.

Light from beam 14 is scattered from area S. Scattered light 18 is detected at an imaging detector 20. Imaging detector 20 may, for example, comprise a digital camera or a video camera. The digital camera may have a CCD array, active pixel sensor or other suitable imaging light detector. The optical axis of imaging detector 20 may be at an angle φ to the normal to area S that is similar to or the same as angle θ.

Apparatus 10 may include other optical components in the path of beam 14 such as diaphragms, mirrors, lenses, other devices that may be used to control, focus, collimate and/or regulate the intensity of a light source, or the like. Any suitable optical systems may be included in apparatus 10.

FIG. 1A shows apparatus 10A according to an alternative embodiment of the invention wherein light beam 14 is carried from light source 12 in an optical light guide and scattered light 18 is carried to an imaging detector 20 in another optical light guide. In the illustrated embodiment, light is carried from light source 12 and directed onto surface S by an inner optical fibre 32A of a light guide assembly 32 and scattered light 18 is collected and delivered to imaging detector 20 by an outer light guide 32B of light guide assembly 32. Light guide 32A may comprise a single mode optical fibre or a multimode optical fibre for example. Light guide 32B may comprise a random fiber bundle or a coherent fiber bundle. In some embodiments, light guide 32A comprises one or more fibres within a coherent bundle and light guide 32B is made up of other fibres within the same coherent fibre bundle. In such cases it is preferred that the one or more fibres that make up light guide 32A be near the centre of the bundle.

A light shield 33 supports the end of light guide assembly 32 a known distance from surface S. Light shield 33 may be opaque to block ambient light from being carried to imaging detector 20. Optical fibre 32A and light guide 32B are shown as being coaxial in FIG. 1A. Other arrangements are also possible. For example, optical fibre 32A and light guide 32B may be located beside one another to provide optical paths similar to those provided by the apparatus of FIG. 1.

Since the light in beam 14 contains a range of wavelengths, imaging detector 20 will capture an image made up of speckle patterns for all of the wavelengths of light in beam 14. The speckle patterns will be shifted relative to one another. This will result in a reduction in contrast in the overall speckle pattern. The amount of the reduction in contrast is dependent on the roughness of area S. By measuring the contrast in the image obtained by imaging detector 20, one can estimate the degree of roughness of area S. The physics of speckle patterns is described, for example, in Dainty J. C. Laser Speckle and related topics, Vol. 9 in the series Topics in Applied Physics, Springer-Verlag, New-York, 1984, which is hereby incorporated herein by reference.

Imaging detector 20 is connected to a computer 30. Imaging detector 20 captures one or more frames of the speckle pattern and transfers those frames to computer 30 by way of a suitable interface. Computer 30 executes software 31 that causes computer 30 to analyze the frames to yield a measure of surface roughness. In some embodiments the measure of surface roughness may be computed from a single image of the speckle pattern imaged by imaging detector 20. In other embodiments, the imaging detector 20 captures multiple frames and software 31 causes computer 30 to generate a measure of surface roughness based upon analysis of multiple frames.

If the contrast of the speckle pattern detected at imaging detector 20 is represented by:

$$C = \frac{{σ}_{I}}{ I } \qquad ( 2 )$$

where:
[symbol] I [symbol] is the average intensity in the image obtained by imaging detector 20; and
σ₁=( [symbol] I² [symbol] −<I>²)^{1/2} is the rms intensity deviation of the light imaged at imaging detector 20 (i.e. the standard deviation of the intensity);
   then it can be shown that:

$$C = \frac{1}{{( 1 + {( 4 {σ}_{k} σ )}^{2} )}^{1 / 4}} \qquad ( 3 )$$

where:
σₖ is the rms spectral deviation from the central wavenumber of the light in beam 14 with k=2π/λ; and
σ is the roughness of the surface of area S.
   It can be shown that:

$$C = \frac{1}{{( 1 + {( 3.39 πσ / {L}_{c} )}^{2} )}^{1 / 4}} \qquad ( 4 )$$

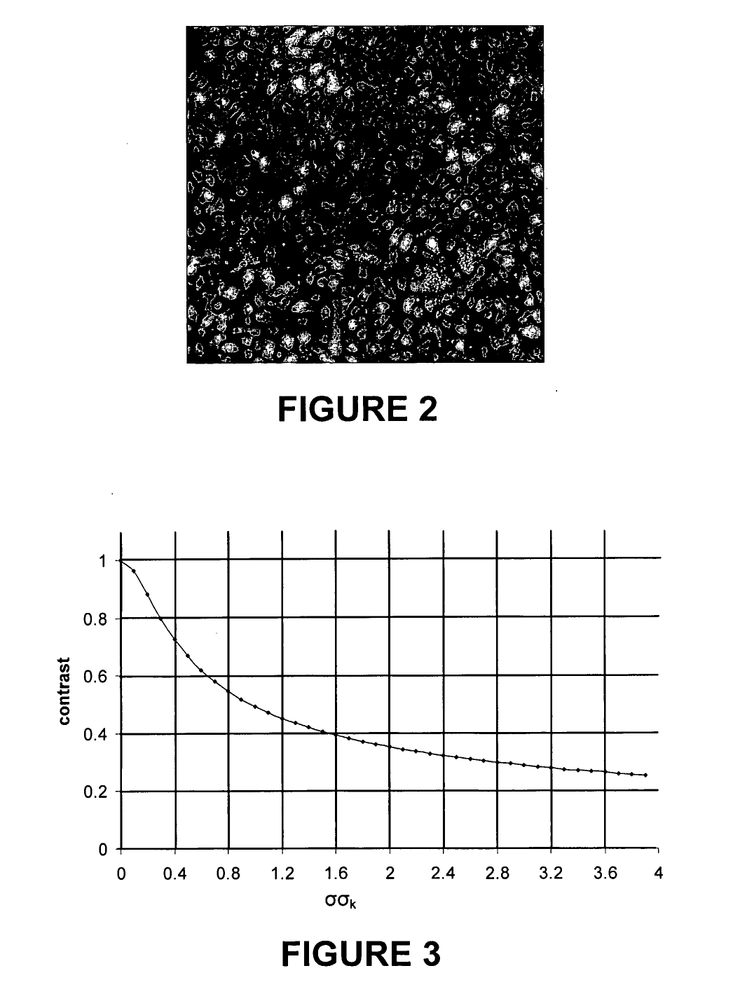

FIG. 3 plots C as a function of σσₖ according to the relationship of Equation (3). One can determine σ, when the spectral range (or equivalently the coherence length L_{c}) of light in beam 14 is known using Equation (3) together with the relation:

$${L}_{C} = \frac{π}{1.18 {σ}_{k}} \qquad ( 5 )$$

Equation (4) can be inverted to give σ as a function of C as follows:

$$σ = \frac{1.18}{4 π} \times {L}_{c} \times \sqrt{( \frac{1}{{C}^{4}} - 1 )} = B \sqrt{( \frac{1}{{C}^{4}} - 1 )} \qquad ( 6 )$$

where B is a calibration parameter that is constant for a particular apparatus as long as the coherence length of the light in beam 14 does not change.

Speckle arises from the constructive and destructive interference of light scattered from different points on area S. Where the coherence length of the light in beam 14 is much smaller than the surface roughness in area S, speckle will not be observed. If the surface roughness is decreased such that it becomes comparable to the coherence length, a speckle pattern will appear.

The contrast of the speckle pattern will increase as the surface roughness decreases. The coherence length of the light in beam 14 determines the range of surface roughness that can be measured. The coherence length is selected to be comparable with the surface roughness to be measured. Consider the case where the coherence length L_{c} is about 200 μm. The condition:

$${σ}_{k} σ = \frac{πσ}{1.18 {L}_{c}} \leq 1 \qquad ( 7 )$$

which can be derived from Equation (3), suggests that the upper limit of roughness that can be detected when L_{c} is about 200 μm is about 75 μm. This value falls in the range of 10 μm to 100 μm which is a range of interest for studies of the roughness of human skin. Larger surface roughness can be measured by using light having a longer coherence length.

The contrast of a speckle pattern may be measured from the data provided by imaging detector 20. Where imaging detector 20 provides image data comprising a pixel value representing the intensity of light detected at each pixel in a rectangular array then the image data may be transferred to a computer 30. The pixel values may be conveniently loaded into a matrix for processing. Any suitable statistical analysis software may be used to obtain mean intensity and rms intensity deviations for rows and columns of the matrix. For example, using the Origin 6.1 software referred to above, the mean intensity and rms intensity deviation may be obtained by applying the “Statistic” function to the rows and columns of the matrix containing the pixel values.

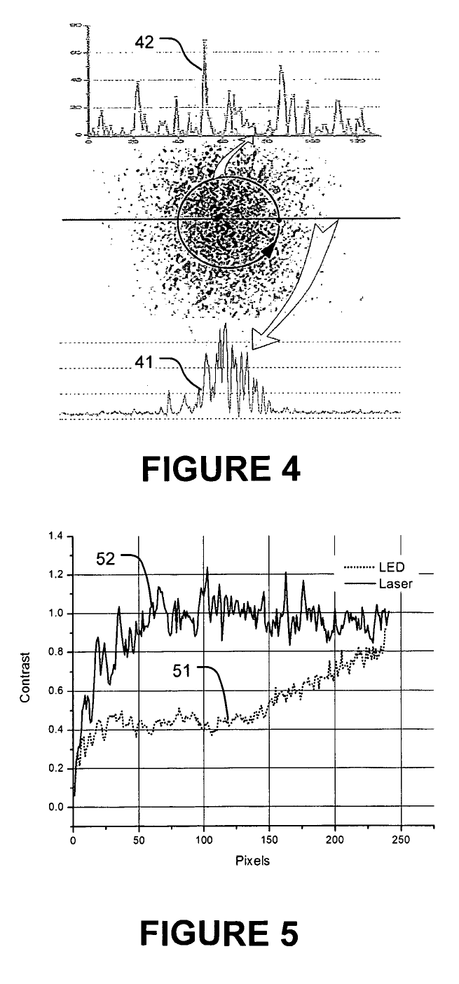

In some cases, finite spatial coherence can cause mean speckle intensity and other characteristics of the speckle pattern to vary with radius. This is illustrated in curve 41 of FIG. 4. When this effect is significant, the calculation of intensity variation by simply averaging over an entire image introduces errors. The inventors have developed a method for determining the speckle pattern contrast in such cases which replaces ensemble averaging with angle averaging. This method is based on the fact that the statistical properties of a speckle pattern do not vary with azimuth angle, as illustrated by curve 42 of FIG. 4.

In the case of a light source characterized by a low-coherence length, the cross-sectional area of the incident beam (in other words, the illuminated spot) can be considered to consist of a number of independent coherent areas (sub-beams). Each individual coherent sub-beam forms an independent speckle pattern. Assuming that the number of independent sub-beams is equal to the ratio of the illuminated area to the coherent area gives:

$$N \approx \frac{{( D / 2 )}^{2}}{{ρ}_{c}^{2}} \qquad ( 8 )$$

where:

### D is the diameter of the light spot on surface S; and

ρ_{c} is the radius of spatial coherence.

For a spatially-incoherent quasi-monochromatic light source with radiating size A, and mean wavelength λ, the radius of spatial coherence is:

$${ρ}_{c} = \frac{λ {Z}_{0}}{A} \qquad ( 9 )$$

where:
Z₀ is the distance between the scattering medium and the light source. A simple formula that expresses contrast in terms of measurable experimental parameters is given by:

$$C = \frac{2 λ {Z}_{0}}{AD} \qquad ( 10 )$$

Accordingly, some embodiments of the invention are configured to perform contrast measurement according to the following procedure:

### 1. Identify a centre point (origin) of the image obtained by imaging detector 20;

### 2. Extract a set of data along a circle centred at the origin and having radius R;

### 3. Calculate the mean value and standard deviation for the set of data and calculate contrast, C(R) for the line.

### 4. Perform steps 2 and 3 for different values of R (for example, start with a value for R and increase R stepwise until increasing R further will expand the circle past the boundary of the image).

Identifying the origin may be performed by any of:
calculating the centre of mass of the image (mass means intensity in this context);
selecting the centre manually, for example, by displaying the image on a computer screen and permitting a user to identify the origin by manipulating a user interface);
detect the centre of mass of a specular (non scattered) component of light; or
a combination of these options.

FIG. 5 shows two examples of contrast radial distributions: Curve 51 shows such a distribution for an LED light source. Curve 52 shows a distribution for a diode laser. In each case, contrast remains relatively constant except in the central zone and very peripheral zones. In the central zones contrast approaches zero due to the presence of a non-scattered specular component. In the peripheral zone of curve 52 contrast goes up with decreasing S/N ratio. Note, that the speckle pattern produced by the diode laser (curve 52) has unit contrast whereas the low-coherence-length LED (curve 51) has a contrast of approximately 0.44 corresponding to the integration of approximately five independent speckle patterns.

Measurements of the contrast of a speckle pattern can be adversely affected by factors such as background light and improperly-set camera black levels. These issues can be addressed by excluding background light and setting black levels so that the values recorded by pixels of imaging sensor 20 do not include a fixed offset or are processed to remove such offset (e.g. an amount equal to the black level may be subtracted from the average intensity values when determining the contrast).

Imaging detector 20 will typically have a digital output. In this case, the gain of imaging detector 20 is preferably adjusted so that the image occupies the whole dynamic range (e.g. 0-255 of gray levels) with no more than a few pixels having maximum values (e.g. 255 units). Setting the gain to a value that is too small or too large results in poor precision in contrast measurements.

To permit the contrast of the speckle pattern to be determined accurately, imaging detector 20 should have a resolution such that individual speckles cover at least several pixels and a field of view large enough to capture a reasonably large number of speckles. If the mean speckle size is too small relative to the pixel size then smoothing will occur which will adversely affect the computation of contrast.

For example, in a prototype embodiment of the invention, imaging detector 20 comprises a CCD camera having a 512×486 pixel sensor (Videoscope International Ltd. model CCD200E). The camera has no objective lens and is arranged at a distance from sample S such that there are about 30 speckles per line (about 900 speckles per frame). This permits the contrast of a speckle pattern to be determined with an accuracy of approximately ±3%. In a prototype embodiment, imaging detector 20 is approximately 260 mm from sample S.

Preferably, the geometry of apparatus 10 is such that the mean speckle diameter at imaging detector 20 is equal to 5 or more times the centre-to-centre pixel spacing of pixels of imaging detector 20. Preferably imaging detector 20 images at least 500, more preferably at least 800 speckles per frame.

The contrast of a speckle pattern and the sizes of individual speckles can be affected by the size of the illuminated spot (e.g. the diameter of beam 14), the angles θ and φ (see FIG. 1) and the distance between area S and imaging sensor 20. In theory, the mean speckle size in the far field is given by:

$$d = \frac{2 \times 1.22 \times Z λ}{D} \qquad ( 11 )$$

where:
d is the mean speckle diameter;

### Z is the distance from the surface at which scattering occurs; and

D is the diameter of the illuminated area on area S (i.e. D is approximately equal to the diameter of beam 14).

Equation (11) can be applied, for example, to the case where Z=260 mm, λ is 658 nm, and D is 3 mm to predict speckles having a diameter d of approximately 123 μm. Where imaging detector 20 is made up of pixels having a size of 8.4 μm per pixel (about 120 pixels/mm) then Equation (11) predicts that the speckles will have a mean diameter of approximately 15 pixels. Similar computations for the case that D=2 mm indicate that the mean speckle diameter should be approximately 25 pixels.

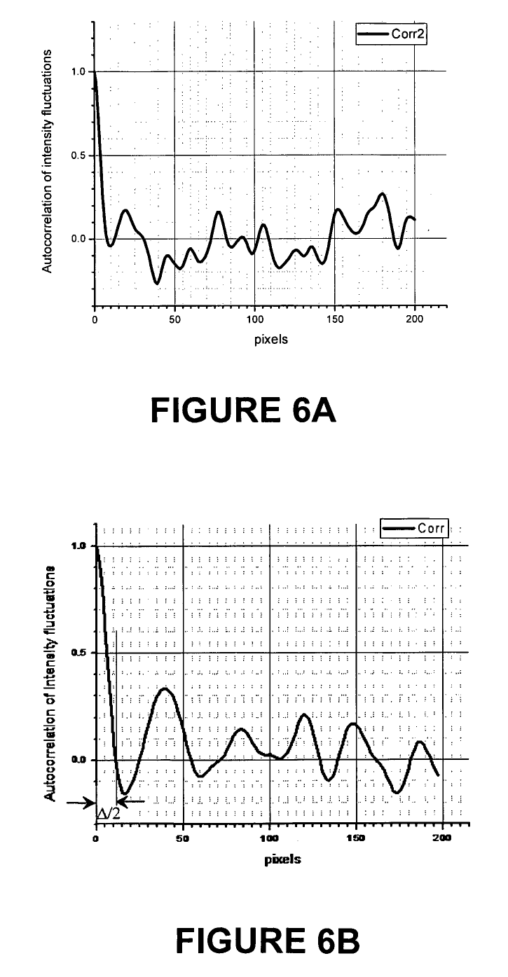

The inventors have conducted experiments to verify Equation (11) using apparatus as shown in FIG. 1 with D=2 mm and D=3 mm. An image of speckles produced using a sandpaper surface having a grit size of 93 μm was analyzed to obtain the mean speckle size. The speckle size can be obtained from a one-dimensional correlation function. FIGS. 6A and 6B are respectively one-dimensional autocorrelation functions for the cases where D=3 mm and D=2 mm. The mean spatial speckle size is determined by measuring the mean width of correlation function. It is enough to calculate one dimensional correlation function to get speckle size. For example, the Correlate function provided in Origin 6.1 data analysis software available from OriginLab Corporation of Massachusetts, USA may be used to calculate the correlation function. The distance Δ (See FIG. 6B) between the origin and the maximum cross-section is one half of the mean speckle size. For the data in FIG. 6B, the mean speckle size is 24 pixels.

The contrast of a speckle pattern can be influenced by geometrical factors. It can be shown that contrast will be reduced by a factor C_{geometry} given by:

$${C}_{geometry}^{2} = \sqrt{\frac{2 ln 2}{π}} \frac{2 {zL}_{c}}{{q}^{2}} = 0.664 \frac{2 {zL}_{c}}{{q}^{2}} \qquad ( 12 )$$

where:
z is the distance from surface S to imaging detector 20; and,
q is the radius of the light spot produced by beam 14 on surface S.

### For example, if L_{c}=10μ, z=50 mm, and q=1 mm then C_{geometry}=0.82.

Equation (12) assumes that:

$${q}^{2} >> \frac{2 \sqrt{π} z \sqrt{1 + {( 4 {σ}_{k} σ )}^{2}}}{{σ}_{k}^{2}} \qquad ( 13 )$$

In some embodiments of the invention, C_{geometry} is taken into account in determining surface roughness. This can be done by dividing the observed contrast by C_{geometry} to yield a value for C which can be used in Equation (3) or (4) above to solve for σ. In general, where the geometrical factors are constant then compensation for the geometrical factors represented by C_{geometry} is included in the overall calibration constant B.

Where area S is an area of a person's skin or another material that is not opaque to the light in beam 14 then it is desirable to remove contributions to the speckle pattern from light that penetrates the skin and is scattered at subcutaneous locations. In the illustrated embodiment, apparatus 10 comprises polarizers 22 and 24. Scattering at the skin surface affects the polarization of polarized light differently from scattering at subcutaneous locations. Polarizer 24 is aligned to reject most light scattered at subcutaneous locations while passing light that is scattered at the surface of area S. An additional polarizer may be provided behind polarizer 22 to control the intensity of the illuminating light. In the alternative, the light output of light source 12 may be adjusted to a desired value, or the intensity of light emitted by light source 12 may be controlled by neutral density filters or other devices that may be provided to adjust the intensity of the light in beam 14.

Another way to reduce contributions to the speckle pattern from light that penetrates the skin and is scattered at subcutaneous locations is to chose the wavelength range of the light in beam 14 so that the light does not penetrate very far into the skin. In general, skin is more opaque at shorter wavelengths than it is at longer wavelengths. By using light that has a shorter wavelength (e.g. by choosing light source 12 so that beam 14 is made up of green or blue light) the effect of subcutaneous scattering can be reduced.

Another way to reduce contributions to the speckle pattern from light that penetrates the skin and is scattered at subcutaneous locations is to obtain images with polarizer 24 set at each of two or more angles. The angles are preferably perpendicular to one another. For example, an image in which the contribution from subcutaneous scatterers is reduced can be obtained by computing:

$$\frac{{I}_{\vert \vert} - {I}_{⊥}}{{I}_{\vert \vert} + {I}_{⊥}} \qquad ( 14 )$$

where:

### I_{ν} and I_{⊥} are the intensities measured with polarizer 24 in two orthogonal positions.

Contributions to a speckle pattern by internally-scattered optical radiation can also be reduced by coating the skin surface with a solution or coating that is strongly absorbing at the wavelength of the optical radiation. Such a solution or coating can block subcutaneously scattered radiation from contributing significantly to a speckle pattern. The coating could also have very high reflectivity so that the optical radiation will not penetrate into the skin. For example, the coating may comprise a metallic paint such as the metallic silver acrylic paint available from Delta Technical Coating, Inc. of California, USA. The coating should be applied in such a manner that it does not fill in rugosities of the skin so as to affect the surface roughness.

A problem with measuring the roughness of skin is that skin cannot be relied upon to stay completely stationary. This problem can exist with other surfaces that move or vibrate. Movement of area S can cause the speckle pattern detected at imaging detector 20 to become blurred. This can be addressed by providing an imaging detector 20 that acquires images of the speckle pattern during a short exposure time. For example, imaging detector 20 may be controlled to provide a short image acquisition time and/or a mechanical shutter (not shown) may be provided to limit the exposure time. In the case of skin, it is desirable to obtain an image of a speckle pattern during an exposure time that is less than 2 ms and preferably less than 1 ms.

In the alternative, or in addition, light source 12 may be pulsed or a shutter may be provided in the path of beam 14 so that light is only projected onto imaging detector 20 for a short time.

A roughness standard 28 may be used to calibrate apparatus 10. Roughness standard 28 may be connected to apparatus 10 by a linkage 29 that permits roughness standard 28 to be stored out of the way during normal use of apparatus 10 and moved into place at the same location as area S for calibrating apparatus 10. Roughness standard 28 has a known roughness. Apparatus 10 can be calibrated by determining the contrast for a speckle pattern produced when roughness standard 28 is illuminated by beam 14. The known surface roughness and contrast can be used to obtain the parameter B of Equation (6) above.

To demonstrate the operation of apparatus 10, the inventors have measured the contrast of speckle patterns produced when various grades of sandpaper that exhibit varying degrees of surface roughness are placed at area S. The mean diameter of sand grains in the different grades of sandpaper ranged between 25 μm and 268 μm. To avoid effects caused by internal reflection within sand grains and reflections from the paper base, each sandpaper sample was coated with aluminum metallic paint. Table I shows results of these trials.

TABLE I
Speckle pattern contrast for sand paper samples for illuminated
spot sizes of 3 mm and 2 mm.

| | | | Mean | | | Mean |
|---|---|---|---|---|---|---|
| | Contrast | | Intensity | Contrast | | intensity |
| Grain | 3 mm | | 3 mm | 2 mm | | 2 mm |
| size (μm) | spot | Error | spot | spot | Error | spot |
| 25 | 1.01 | 0.08 | 24.25 | 1.01 | 0.08 | 29.65 |
| 60 | 0.92 | 0.07 | 34.42 | 0.9 | 0.09 | 51.45 |
| 93 | 0.98 | 0.08 | 27.25 | 0.98 | 0.08 | 32.32 |
| 116 | 1 | 0.09 | 27.85 | 0.97 | 0.09 | 30.69 |
| 141 | 0.97 | 0.08 | 28.2 | 0.96 | 0.1 | 24.04 |
| 268 | 0.91 | 0.09 | 13.06 | 0.89 | 0.11 | 17.93 |

The inventors have also measured the contrast of speckle patterns produced by metal roughness standards having roughnesses in the range of 0.8 μm to 25.4 μm. Results of these experiments are shown in Table IA.

TABLE IA
Measured Roughness for Metal Standards

| Object | Roughness (μm) | Contrast |
|---|---|---|
| #32 | 0.8 | 0.96 ± 0.04 |
| #63 | 1.6 | 1.04 ± 0.07 |
| #125 | 3.17 | 0.97 ± 0.03 |
| #250 | 6.35 | 0.89 ± 0.04 |
| #500 | 12.7 | 0.73 ± 0.08 |
| #1000 | 25.4 | 0.67 ± 0.08 |

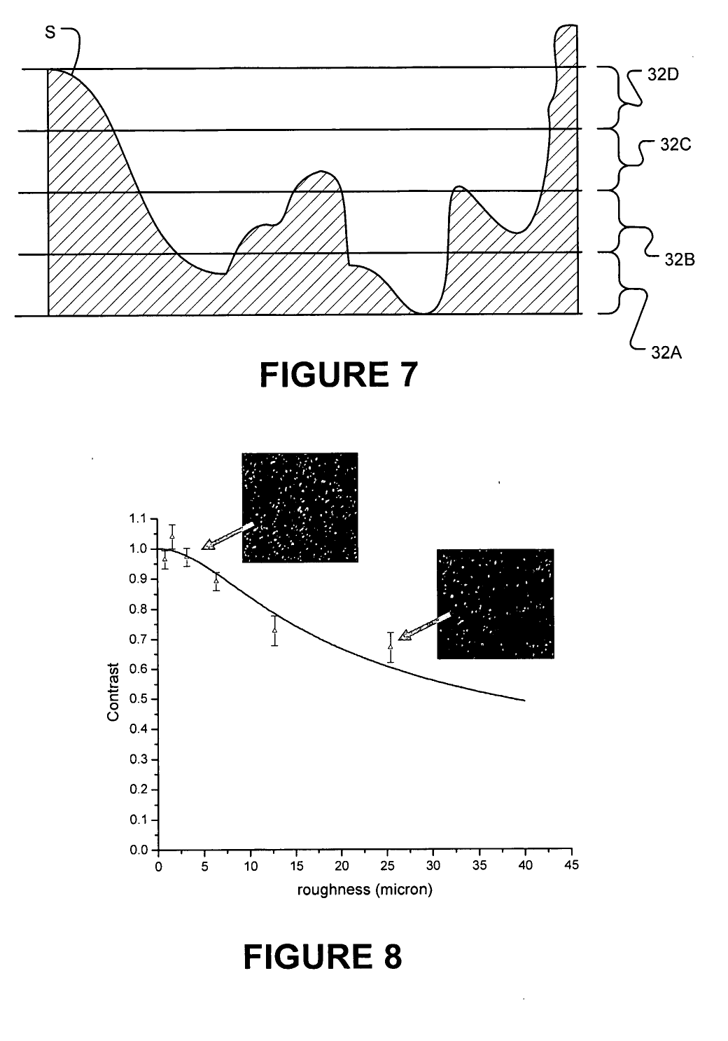

While the inventors, do not wish to be bound by any particular theory of operation, it is believed that the mechanism by which contrast is reduced as surface roughness increases can be visualized by considering the speckle pattern created in the apparatus of FIG. 1 to be made up of independent speckle patterns arising from different layers of the surface. FIG. 7 shows a case where the illuminating light has a coherence length that is less than the height of surface roughness features. Layers 32A through 32D each have a thickness equal to an effective coherence length of the illuminating radiation. The effective coherence length is typically approximately ⅜ times L_{c}. Each layer 32A to 32D can be considered to create an independent speckle pattern. If the contrast of the speckle pattern of each layer is equal to one then the speckle pattern resulting from the combination of N independent speckle patterns is expected to have a contrast given by:

$$C = \frac{1}{\sqrt{N}} \qquad ( 15 )$$

in the case where all of the independent speckle patterns have equal mean intensities.

The inventors have tested the relationship of Equation (15) by making a target consisting of several layers of sandpaper having 25 μm grit size. The layers were at different distances from light source 12 (separated by about 600 μm) so that each layer produced an independent speckle pattern that contributed to the overall speckle pattern detected by imaging detector 20. The layered surface was illuminated with a beam 14 having a diameter of 1.5 mm. The layered surface was located at a distance of 285 mm from the imaging sensor. The results of these measurements are shown in Table II.

TABLE II
Contrast of Speckle Pattern from Multi-Layer Surface

| Number of Layers | Theoretical | Measured | |
|---|---|---|---|
| (N) | contrast | contrast | Error |
| 1 | 1 | 0.99 | 0.02 |
| 2 | 0.71 | 0.75 | 0.05 |
| 3 | 0.58 | 0.63 | 0.04 |

FIG. 8 is a graph showing contrast as a function of surface roughness for various materials. A red diode laser was used as light source 12. The points having error bars correspond to sandpaper of various grades. The points without error bars correspond to metal roughness standards. The curve indicates the best fit of the theoretical formula of Equation (4) to the data of FIG. 8. Two speckle patterns corresponding to the points indicated by arrows are also shown in FIG. 8.

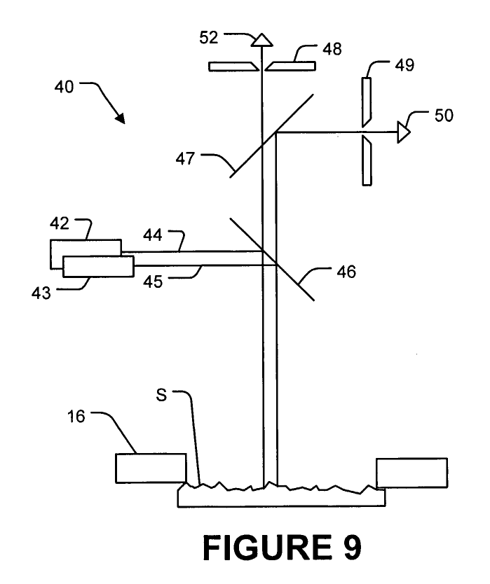

FIG. 9 shows alternative apparatus 40 for measuring surface roughness in which an area S of skin (or another surface) is illuminated by light having two discrete wavelengths. Area S is illuminated by light beams 44 and 45 emitted respectively by two light sources 42 and 43. A single light source that provides light having two suitable wavelengths can be used in the alternative.

Each of beams 44 and 45 is reflected toward area S by a semi-transparent mirror 46. The light is scattered by the surface in area S to yield speckle patterns. An independent speckle pattern is formed at each wavelength. Light from the centre of each speckle pattern is directed to a separate light detector. Light from the speckle pattern caused by beam 45 is reflected by a dichroic mirror 47 through an aperture 49 to a light detector 50. Light from the speckle pattern caused by beam 44 passes through semi-transparent mirror 46, dichroic mirror 47 and aperture 48 to a second light detector 52.

The rms difference between the normalized speckle intensity distributions resulting from beams 44 and 45 can be expressed as:

$$W ( {k}_{1} , {k}_{2} ) = { [ \frac{I ( {k}_{1} )}{ I ( {k}_{1} ) } - \frac{I ( {k}_{2} )}{ I ( {k}_{2} ) } ]}^{2} {1 / 2}^{} \qquad ( 16 )$$

where: [symbol]
. . . [symbol] indicates ensemble averaging;
k₁ and k₂ represent the wave vectors of beams 44 and 45 respectively; and,

### I represents the measured on-axis (θ=0) intensity of a speckle intensity distribution.

The relationship between the surface roughness and the difference in the intensity distributions of the two speckle patterns can be expressed as:

$$W ( {k}_{1} , {k}_{2} ) = \sqrt{2 ( 1 - exp ( \frac{- {{σ}^{2} ( {k}_{1} - {k}_{2} )}^{2}}{4} ) )} \qquad ( 17 )$$

where, on-axis, k₁=2π/λ₁ and k₂=2π/λ₂.

W can be measured by making sufficiently many measurements of the signals from light detectors 50 and 52, while moving light beams 44 and 45 relative to area S, to obtain statistically valid measurements of [symbol] I(k₁) [symbol] and [symbol] I(k₂) [symbol] .

Preferably the wavelengths of beams 44 and 45 are selected such that:

σ|(k₁−k₂)|≦1 (18)

where σ is the roughness of the surface to be measured. For the measurement of surfaces having roughnesses greater than a few μm the difference between the wavelengths of beams 44 and 45 should be very small.

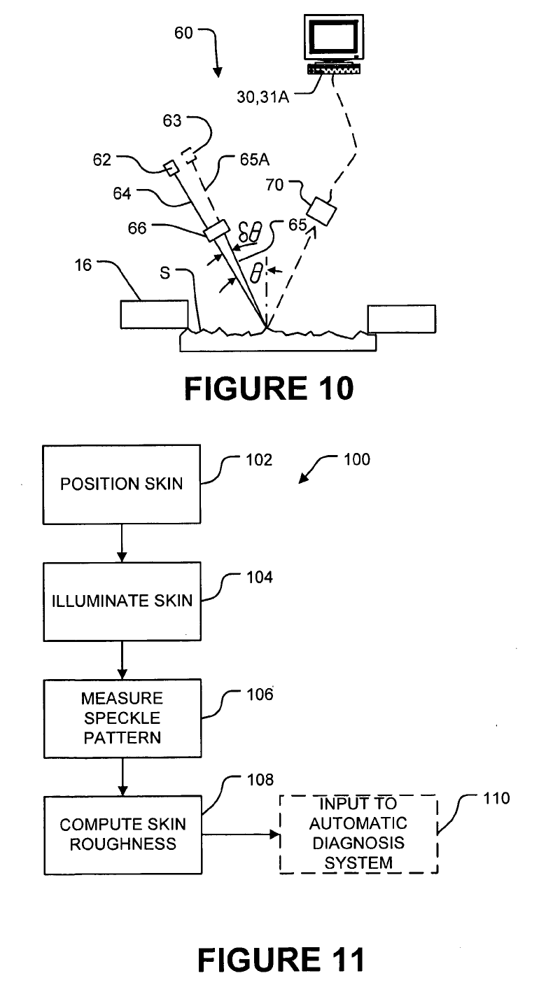

FIG. 10 shows another apparatus 60 that may be used for measuring the roughness of skin or other surfaces. Apparatus 60 operates according to principles described in Leger D. et al. Optical surface roughness determination using speckle correlation technique, Applied Optics 14 (4), pp. 872-877, (1975).

Apparatus 60 includes a light source 62 that issues a beam of light 64 toward a surface S being studied. Surface S may be, for example, the surface of a subject's skin. Apparatus 60 includes a deflection mechanism 66 that can be operated to change the angle θ at which beam 64 is incident on surface S by an amount δθ (the beam incident at the changed angle is identified by the reference numeral 65. As in the embodiments above, a support 16 is provided to facilitate placing a surface to be studied (such as a skin surface) at a known location.

As an alternative to the provision of a mechanism 66, apparatus 60 could have a second light source 63 oriented to direct a second beam of light 65A onto surface S at an angle that differs from θ by an amount δθ. Light source 63 should produce optical radiation that is the same as the optical radiation produced by light source 62.

An imaging light sensor 70 records speckle patterns resulting from the incidence of each of beams 64 and 65. Imaging light sensor 70 may comprise photographic film or an array of light sensors such as a CCD, CMOS or APS array. The two speckle patterns are added together. This may be done, for example, by recording the two speckle patterns on the same piece of film or using the same light-sensing array, either sequentially or simultaneously, or by separately acquiring and adding together pixel values in images of the two speckle patterns.

For small values of δθ the speckle pattern from beam 65 will be a modified version of the speckle pattern from beam 64. In general, the differences between the two speckle patterns will include translations and changes in the distribution of light intensity (decorrelation).

One way to obtain information about the roughness of surface S is to obtain the Fourier transformation of the combined speckle patterns. The Fourier transformation may be performed in the optical domain or by computation from the measured pixel intensities. The Fourier transformed combined image will include Young's interference fringes. The visibility V of those fringes is given by:

$$V = \frac{{I}_{max} - {I}_{min}}{{I}_{max} + {I}_{min}} = exp ( - {[ \frac{2 π}{λ} σsin ( θ ) δθ ]}^{2} ) \qquad ( 19 )$$

where:

### Iₘₐₓ and Iₘᵢₙ are respectively the maximum and minimum intensities of the Young's fringes;

λ is the wavelength of light in beams 64 and 65;
σ is the roughness of surface S; and
θ and δθ are as shown in FIG. 10.

The range of surface roughness that can be measured using apparatus 60 is dependent upon the geometry and the characteristics of the light in beams 64 and 65. It is desirable that V is in the range of 0.1 to 0.8 to obtain the most accurate measurements. Table III gives some example operating conditions and the corresponding range of surface roughness that can be measured for V between 0.1 and 0.8.

TABLE III

| λ | θ (degrees) | δθ (degrees) | range of σ (μm) |
|---|---|---|---|
| 632 | 45 | 0.5 | 10 to 30 |
| 632 | 45 | 2 | 3 to 13 |

It can be seen that smaller values for δθ permit measurement of larger roughness. A small value for δθ also reduces noise by reducing the linear shift between the two speckle patterns in the registration plane (i.e. the plane of imaging detector 70). The linear shift, Δ, is given by:

Δ=z cos θδθ (20)

If the ratio of the size of imaging detector 70 to Δ is too small then the contrast of Young's fringes will be reduced because some speckles of the first speckle pattern will fall outside of the imaging detector 70 in the second speckle pattern and vice versa. As a result, not all speckles will have a pair in the image data from imaging detector 70. Such non-paired speckles will create noise during signal development and decrease the contrast of Young's fringes.

It is generally desirable to maintain a ratio of Δ/D in excess of 6 and preferably in excess of 8, where D is a dimension of imaging detector 70. For example, Using z=70 mm, θ=45, and δθ=30′ results in Δ=0.52 mm. If imaging detector 70 is a CCD camera or the like having a 5.2 mm by 5.2 mm CCD array, the ratio Δ/D=10. In this case 10 Young's interference fringes will be observed. 10 fringes is sufficient to provide good precision for calculations of V. Once V has been determined, surface roughness can be evaluated from Equation (19).

It is optionally possible to record three or more speckle patterns, each generated by optical radiation having a different angle if incidence θ. Young's fringes may be obtained by combining any two of such speckle patterns. The visibility of the Young's fringes may be computed for any one or more of the resulting combinations. Measures of the surface roughness may be obtained from the visibility of the Young's fringes as described above.

Signals may be output from imaging detector 70 and provided to a computer 30 as image data by way of a suitable interface. Computer software 31A running on computer 30 processes the image data to compute a value for the surface roughness, as described above.

It can be appreciated that the systems and methods described herein may be used to measure surface roughness of biological samples, such as skin, or of other samples in real time. Such systems and methods may be used in manufacturing processes, quality control processes or processes of applying surfaces to materials. The systems and methods may be used to provide feedback, including real time feedback, in manufacturing processes, coating processes or quality control processes.

FIG. 11 is a flow chart illustrating a method 100 for measuring skin roughness. Method 100 begins at block 102 by placing an area of skin of interest at a point that can be illuminated with a light source to generate a speckle pattern as described above. Block 102 may comprise placing a part of a subject's body against a positioning member 16 as described above. Where apparatus according to the invention has a movable sensing head, which may be, for example, in the form of a hand-held wand, block 102 may comprise positioning the sensing head against the area of skin of interest.

In some embodiments, block 102 comprises displaying an image of an area of skin together with indicia indicating a position to which the illumination may be delivered so that a particular lesion or other skin portion of interest may be studied. To facilitate this, apparatus according to the invention may include a separate camera and display or an imaging sensor, such as imaging sensor 20 may be placed in a mode in which it obtains an image of the skin surface. This may involve adjusting imaging optics or inserting an objective lens in the optical path between imaging detector 20 and the skin surface.

In block 104 the skin surface is illuminated with a light beam. Illumination of the skin surface generates at least one speckle pattern. In some embodiments, block 104 comprises illuminating the skin surface with optical radiation having a coherence length comparable to the expected roughness of skin. For example, the coherence length may be less than 300 μm or, in some embodiments, in the range of 20 μm to 250 μm.

In block 106 measurements are obtained of light intensity in the speckle pattern.

In block 108 data from the measurements is processed in a digital computer or in a logic circuit or in a combination thereof to yield surface roughness information characterizing a surface roughness of the skin.

Optionally, in block 110 the surface roughness information is provided as an input to an automatic diagnostic system. The automatic diagnostic system generates a diagnosis on the basis of the surface roughness information taken in combination with other information provided as inputs to the automatic diagnostic system. For example, an automatic diagnostic system attempting to determine whether a lesion is seborrheic keratosis or malignant melanoma may receive an input containing information specifying surface roughness of the lesion from a roughness-measurement system as described herein. Since roughness is diagnostic for malignant melanoma, the automatic diagnostic system may increase a probability of a diagnosis of malignant melanoma by an amount in inverse proportion to the measured roughness, as indicated by the input, or by some amount in response to the measured roughness being below a threshold.

In some embodiments the automatic diagnostic system has a function for distinguishing between seborrheic keratosis, dysplastic nevus, and melanoma. These conditions are sometimes difficult to differentiate clinically. Roughness measurements are useful in such diagnosis because these different types of lesions are generally characterized by different surface roughnesses. The order of surface roughness of these three types of lesions is: skin affected by seborrheic keratosis tends to be rougher than skin affected by dysplastic nevus which tends to be rougher than skin affected by melanoma.

In some embodiments the automatic diagnostic system has a function for distinguishing between squamous cell carcinoma and various precancerous conditions such as warts, actinic keratosis, and Bowen disease. Roughness measurements are useful in such diagnosis because these different types of lesions are generally characterized by different surface roughnesses. The order of roughness for this cluster of lesions is: skin affected by warts tends to be rougher than skin affected by actinic keratosis which tends to be rougher than skin affected by Bowen disease which tends to be rougher than skin affected by squamous cell carcinoma.

Selected methods as described herein can be used to measure the coherence length of light sources. Coherence length is an important parameter in many optical systems. Coherence length can be affected by the operating environment of a light source. The coherence-length measuring aspects of the invention may be applied to determine the coherence length of light from a light source in its operating environment.

Coherence length can be evaluated by observing speckle patterns that arise when light is scattered from a set of standard references having different known surface roughness. The roughness of the standard should be in the same range as the coherence length of the light source. For the measurement of longer coherence lengths, standards that are very rough may be provided. In some embodiments, such standards comprise porous media or media having needle-like projections.

Coherence-length measurements may be performed with a backscattering geometry or a transmission geometry. In a backscattering geometry the standards are reflective. Light reflected from the surface of the standard creates a speckle pattern. In a transmission geometry, the standard may comprise a transparent material having a rough surface such as a glass standard. Light that passes through the standard and is scattered at the rough surface yields a speckle pattern. In either case, the speckle pattern is analyzed to obtain a measurement of the coherence length of the light given the known roughness of the standard.

For example, the coherence length of the light in beam 14 (see FIG. 1) can be determined if the roughness of the surface with which beam 14 interacts to create a speckle pattern is known. The contrast vs. roughness function of Equation (4) can be fitted to the experimental points for the six samples in Table IA to yield the average parameter β=3.39π/L_{c}. In one case, light source 12 comprised a SLED (SLD-3P-680, B&W TEK Inc, USA). The fitting resulted in a value β3=0.242 μm⁻¹, corresponding to a coherence length of 44 μm. This result is close to the theoretical value of 50 μm as calculated using Equation (1) and the given spectral characteristics (λ=683.6 nm, Δλ=9.5 nm) for the SLED.

The invention may be embodied in a system that includes a computer 30 and software which causes the computer to analyze an image of a speckle pattern originating from a surface having a known roughness and calculate the linewidth of the light source (or, equivalently, the coherence length of the light source) from the contrast of the speckle image. This calculation may be performed by solving Equation (6), or a mathematical equivalent thereof, for L_{c}.

Certain implementations of the invention comprise computer processors which execute software instructions which cause the processors to perform a method of the invention. For example, one or more processors in a computer may implement the method of FIG. 11 executing software instructions in a program memory accessible to the processors. The invention may also be provided in the form of a program product. The program product may comprise any medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable signals on the program product may optionally be compressed or encrypted.

Where a component (e.g. a light source, light detector, software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof. For example:
a two-dimensional imaging sensor 20 may comprise a CCD camera or any other sensor capable of detecting the optical radiation. For example, an imaging sensor 20 may comprise an array of CMOS, ICCD, CID sensors or the like.
a light source may be made up of two or more light sources having outputs that are combined to provide optical radiation for purposes of the invention.
   Accordingly, the scope of the invention is to be construed in accordance with the substance defined by the following claims.

What is claimed is:

1. A method for measuring roughness of an area of a biological surface in vivo, the method comprising:
illuminating an area of a biological surface of a subject with coherent optical radiation and allowing the optical radiation to scatter from the area of the biological surface to yield a speckle pattern;
making measurements of intensity of the optical radiation in the speckle pattern; and,
based upon results of the measurements, computing a measure of roughness of the area of the biological surface.

2. A method according to claim 1 wherein making measurements of intensity of the optical radiation in the speckle pattern comprises imaging light scattered from the area of the biological surface onto a two-dimensional imaging detector.

3. A method according to claim 2 wherein the imaging detector comprises an array of pixels and a mean size of speckles at the imaging detector of speckles in the speckle pattern is at least 5 times greater than a center-to-center spacing of adjacent pixels in the array.

4. A method according to claim 2 comprising imaging at least 500 speckles onto the imaging detector.

5. (canceled)

6. A method according to claim 2 wherein computing the measure of roughness of the area of the biological surface comprises determining a contrast of the speckle pattern and computing the measure of roughness of the area of the biological surface based on the contrast of the speckle pattern.

7. A method according to claim 6 wherein the measure of roughness is proportional to:
$$\sqrt{( \frac{1}{{C}^{4}} - 1 )}$$
or a mathematical equivalent thereof, where C is the contrast of the speckle pattern.

8. A method according to claim 7 wherein the measure of roughness is given by:
$$σ = B \sqrt{( \frac{1}{{C}^{4}} - 1 )}$$
where σ is the measure of roughness and B is a calibration constant.

9. A method according to claim 8 comprising computing a value for the calibration constant by:
placing a roughness standard having a known roughness in place of the area of the biological surface;
illuminating the roughness standard with the optical radiation to yield a standard speckle pattern;
computing a contrast of the standard speckle pattern; and,
calculating a value for the calibration constant from the known roughness and the contrast of the standard speckle pattern.

10. (canceled)

11. A method according to claim 8 wherein determining the contrast of the speckle pattern comprises identifying a center of the speckle pattern and computing the contrast based on values lying within an annular ring around the center of the speckle pattern.

12. A method according to claim 6 wherein a wavelength of the optical radiation is shorter than 600 nm.

13. A method according to claim 6 wherein the optical radiation comprises green or blue light.

14. A method according to claim 2 wherein the optical radiation is polarized and making measurements of intensity of the optical radiation in the speckle pattern comprises making measurements of intensity of a component of the optical radiation in the speckle pattern, the component having a predetermined polarization.

15. A method according to claim 2 wherein the optical radiation is polarized and making measurements of intensity of the optical radiation in the speckle pattern comprises making measurements of intensity of at least two components of the optical radiation, the two components having different polarizations.

16. A method according to claim 15 wherein the two polarizations are substantially perpendicular.

17. A method according to claim 16 wherein computing a measure of roughness of the area of the biological surface comprises computing the value A given by:
$$A = \frac{{I}_{\vert \vert} - {I}_{⊥}}{{I}_{\vert \vert} + {I}_{⊥}}$$
or a mathematical equivalent thereof and calculating the measure of roughness based on A.

18. A method according to claim 2 wherein the optical radiation has a coherence length of 500 μm or less.

19-20. (canceled)

21. A method according to claim 1 wherein making measurements of intensity of the optical radiation in the speckle pattern is performed during an exposure time of 2 ms or less.

22-25. (canceled)

26. A method according to claim 1 wherein illuminating an area of the biological surface of a subject with coherent optical radiation comprises illuminating the area of the biological surface with optical radiation having first and second distinct wavelengths and, separately for each of the wavelengths, obtaining multiple measurements of an intensity at a point in the speckle pattern.

27. A method according to claim 26 comprising ensemble averaging the multiple measurements for each of the first and second wavelengths.

28. A method according to claim 27 wherein the following inequality between the first and second wavelengths and the roughness of the area of the biological surface holds:
$$σ \vert \frac{2 π}{{λ}_{1}} - \frac{2 π}{{λ}_{2}} \vert \leq 1$$
where λ₁ and λ₂ are respectively the first and second wavelengths and σ is the roughness of the area of the biological surface.

29. A method according to claim 26 comprising moving the area of the biological surface relative to a source of the optical illumination between taking the multiple measurements.

30. A method according to claim 26 comprising computing the value:
$$W ( {k}_{1} , {k}_{2} ) = { [ \frac{I ( {k}_{1} )}{ I ( {k}_{1} ) } - \frac{I ( {k}_{2} )}{ I ( {k}_{2} ) } ]}^{2} {1 / 2}^{} \qquad ( 4 )$$
or a mathematical equivalent thereof, where: [symbol]
. . . [symbol] indicates ensemble averaging;
k₁ and k₂ represent the wave vectors of the optical radiation at the first and second wavelengths respectively; and, I(k₁) is the measured intensity of the speckle intensity distribution at the first wavelength and I(k₂) is the measured intensity of the speckle intensity distribution at the second wavelength and computing the roughness measure based on the value computed for W.

31. A method according to claim 1 wherein illuminating the area of the biological surface comprises illuminating the area of the biological surface with the optical radiation incident from first and second angles.

32. A method according to claim 31 comprising, obtaining an image comprising Young's fringes resulting from the combination of first and second speckle patterns, the first speckle pattern resulting from illumination at the first angle and the second speckle pattern resulting from illumination at the second angle.

33. A method according to claim 32 comprising computing a visibility of a Young's fringe and wherein computing the measure of roughness of the area of the biological surface is based at least in part on the visibility.

34. A method according to claim 33 wherein computing the visibility comprises computing a value V given by:
$$V = \frac{{I}_{max} - {I}_{min}}{{I}_{max} + {I}_{min}}$$
or a mathematical equivalent thereof, where Iₘₐₓ and Iₘᵢₙ are respectively maximum and minimum intensities for the Young's fringe.

35. A method according to claim 33 wherein computing the measure of roughness is based on visibilities of a plurality of Young's fringes.

36. A method according to claim 35 wherein obtaining an image comprising Young's fringes comprises imaging the first and second speckle patterns at an imaging detector, wherein, at the imaging detector, the Young's fringes have a spacing that is less than ⅛ of a width of an area of the imaging detector that is responsive to the optical radiation.

37. A method according to claim 1 comprising aligning the optical radiation to be incident on a lesion on the area of the biological surface.

38. A method according to claim 37 wherein aligning the optical radiation comprises displaying an image of the area of the biological surface together with indicia indicating a point at which the optical radiation will illuminate the biological surface.

39. A method according to claim 37 comprising performing the method with the optical radiation aligned to be incident on the lesion to obtain a first measure of roughness and repeating the method with the optical radiation aligned to be incident on a part of the biological surface off of the lesion to obtain a second measure of roughness.

40. A method according to claim 2 comprising providing the roughness measure as an input to an automatic diagnostic system wherein the biological surface comprises skin.

41. (canceled)

42. A method according to claim 41 comprising, in the automatic diagnostic system, increasing a probability of the skin being affected by malignant melanoma in response to the roughness measure indicating a roughness being below a threshold roughness.

43. (canceled)

44. A method according to claim 41 comprising, in the automatic diagnostic system, increasing a probability of the skin being affected by seborrheic keratosis in response to the roughness measure indicating a roughness being above a threshold roughness.

45. (canceled)

46. A method according to claim 41 wherein the automatic diagnostic system comprises a function for distinguishing between seborrheic keratosis, dysplastic nevus, and melanoma and the method comprises providing the roughness measure, or a value derived from the roughness measure, as an input to the function.

47. A method according to claim 41 wherein the automatic diagnostic system has a function for distinguishing between squamous cell carcinoma and one or more conditions selected from the group consisting of: warts, actinic keratosis, and Bowen disease and the method comprises providing the roughness measure, or a value derived from the roughness measure, as an input to the function.

48. A method according to claim 47 comprising, in the automatic diagnostic system, increasing a probability of the skin being affected by squamous cell carcinoma in response to the roughness measure indicating a roughness being below a threshold roughness.

49-55. (canceled)

56. Apparatus for measuring the roughness of a biological surface, the apparatus comprising a light source emitting optical radiation having a coherence length of 300 μm or less;
an imaging detector located to detect the optical radiation after the optical radiation has been scattered from a biological surface; and,
a processor connected to receive image data from the imaging detector and configured to:
compute a contrast of a speckle pattern in the scattered optical radiation; and,
compute a roughness of the biological surface from the contrast.

57. Apparatus according to claim 56 comprising an opaque light shield extending around the light source and imaging detector, the light shield having an edge that can be brought to bear against the biological surface.

58. Apparatus according to claim 56 comprising a support surface located in a known relationship to the light source and imaging detector wherein, with the support surface bearing against a biological surface, optical radiation from the light source can illuminate an area of the biological surface to yield a speckle pattern detectable by the imaging detector.

59-61. (canceled)

62. Apparatus according to claim 58 comprising a first light guide providing an optical path for carrying the scattered optical radiation to the imaging detector and a second light guide disposed to carry the optical radiation from the light source and to emit the optical radiation to illuminate a biological surface to be studied.

63. (canceled)

64. Apparatus according to claim 62 wherein the second light guide is coaxial with the first light guide.

65. Apparatus according to claim 63 wherein ends of the first and second light guides are substantially equidistant from a biological surface to be studied.

66-71. (canceled)

72. Apparatus according to claim 56 wherein the light source has a variable coherence length.

73. Apparatus according to claim 72 wherein the light source comprises a plurality of narrow-band filters each having a different bandwidth and being disposed to be selectively interposed in a path of the optical radiation.

74. (canceled)
